(19) United States
(12) Patent Application Publication
(10) Pub. No.: US 2017/0043125 A1
(43) Pub. Date: Feb. 16, 2017

(54) SYSTEM AND METHOD FOR LOCAL DELIVERY OF THERAPEUTIC COMPOUNDS

(71) Applicant: The Children's Hospital of Philadelphia, Philadelphia, PA (US)

(72) Inventors: Robert J. Levy, Merion Station, PA (US); Mark Battig, Philadelphia, PA (US); Michael Chorny, Huntingdon Valley, PA (US)

(21) Appl. No.: 15/337,549

(22) Filed: Oct. 28, 2016

Related U.S. Application Data
(63) Continuation-in-part of application No. PCT/US2015/027916, filed on Apr. 28, 2015.
(60) Provisional application No. 61/984,938, filed on Apr. 28, 2014.

(51) Int. Cl. A61M 25/00; A61K 47/48

(52) U.S. Cl. CPC A61M 25/0082; A61K 47/48861; A61M 2025/0096

(57) ABSTRACT

An apparatus for preloading magnetic particles or cells onto a medical device includes a device carrier and a particle carrier attached over the device carrier. At least one magnet is inserted into the device carrier, the at least one magnet extending inside a section of the device carrier, over which the particle carrier is attached, to attract magnetic particles or cells to the particle carrier when the apparatus is placed in a suspension of magnetic particles or cells. A method for preloading magnetic particles or cells onto a medical device includes attaching one or more magnets to a device carrier, attaching a particle carrier to the device carrier in proximity to the one or more magnets, and inserting the device carrier with the attached particle carrier and the one or more magnets into a suspension containing magnetic particles or cells to preload the particle carrier with magnetic particles or cells.

### RELATED APPLICATIONS

This application is a continuation-in-part under 35 U.S.C. §365(c) of International Application No. PCT/US2015/027916, filed Apr. 28, 2015, which is related to and claims the benefit of priority under 35 U.S.C. §119 of U.S. Provisional Application No. 61/984,938, filed Apr. 28, 2014. The contents of International Application No. PCT/US2015/027916 and U.S. Provisional Application No. 61/984,938 are incorporated by reference herein in their entireties.

### FIELD

The present invention relates generally to therapeutic treatment of humans and animals, and more specifically to a system and method for delivering therapeutic compounds using a magnetic targeting device that is readily insertable into and removable from the human or animal.

### BACKGROUND

The applicant has developed systems and methods for magnetic targeting of iron oxide-containing, magnetically responsive, biodegradable nanoparticles containing therapeutic agents (MNP) to areas in the body that require treatment. These systems and methods have been developed to deliver MNP in vivo to permanently deployed stents or other implanted devices. The feasibility of this approach has been demonstrated for delivering drugs, gene vectors and cell therapy. Targeting of MNP to permanent stents can be enhanced by application of a relatively uniform magnetic field. The following patents and published patent applications, which describe various aspects of magnetic targeting procedures, are incorporated herein by reference: U.S. Pat. No. 8,562,505, U.S. Pat. No. 7,846,201, U.S. Pub. No. 2009/0216320, U.S. Pub. No. 2010/0260780 and International Pub. No. WO 2004/093643.

The applicant has also developed systems and methods that target MNP to treatment areas in the body using “temporary” magnetic targeting devices, as opposed to stents or other implants. Temporary magnetic targeting devices can be inserted into the area of treatment under a uniform magnetic field, where they can be used to target MNP to the treatment site and then be removed from the treatment site. International Pub. No. WO 2012/061193, which is incorporated by reference herein in its entirety, describes a number of different temporary magnetic targeting devices that can be used. One such device incorporates a magnetic targeting catheter (MTC) featuring an expandable mesh formed of a magnetizable material at the distal end of the catheter. In experiments, the MTC was inserted into an artery and advanced into proximity of a diseased section of the arterial wall. The diseased section of arterial wall was exposed to a uniform magnetic field. The expandable mesh at the distal end of the MTC was expanded into contact with the diseased arterial wall. A suspension containing MNP was then injected through the catheter into the artery. MNP were magnetically targeted to the expanded mesh, thereby directing the MNP into contact with the diseased arterial wall where the MNP were retained.

While MTCs have shown promise in targeting MNP to treatment sites, the applicant has discovered a few challenges in using MTCs. One challenge in particular is targeting MNP to an MTC after the magnetized tip or mesh is deployed in the artery at the treatment site. MNP that are introduced into an artery in the presence of a uniform magnetic field do not always attach to the magnetized mesh. Some MNP can be swept into the bloodstream and flow past the mesh, or be drawn into side branches of the artery where they are drawn away from the treatment site.

To avoid losing MNP in the bloodstream, the applicant has designed MTC devices that are equipped with balloons that can be expanded to temporarily occlude the artery at locations upstream and downstream of the treatment site. By occluding the artery, the balloons temporarily stop blood from flowing past the treatment site, thereby allowing the MNP to be targeted to the mesh in a static environment. Targeting MNP to the mesh while blood flow is halted can reduce the loss of MNP in the artery. Nevertheless, it is undesirable to stop blood flow in an artery for any length of time. In addition, occlusion balloons require the MTC to incorporate special control mechanisms and require additional channels in the catheter body. The additional control mechanisms and multi-channel catheter structure can increase the overall diameter of the catheter, limiting access to small vessels, while also decreasing the flexibility and maneuverability of the device. The additional control mechanisms can also add significant cost to the manufacture of the MTC, as the MTC requires a customized design.

### SUMMARY

Devices and methods are provided for delivering magnetic particles containing a beneficial agent, such as a therapeutic agent or imaging compound, to an area in an animal or human subject. In the case of a therapeutic agent, the devices and methods can be used to deliver magnetic particles containing a therapeutic agent to an area requiring treatment, such as a diseased wall in a blood vessel.

In one embodiment of the invention, an apparatus is used for preloading magnetic particles onto a medical device for delivering the magnetic particles to a target site. The apparatus can include a device carrier comprising a hollow tubular body, and a particle carrier attached over the hollow tubular body of the device carrier. The apparatus can also include at least one magnet inserted into the hollow tubular body of the device carrier, the at least one magnet extending inside a section of the tubular body over which the particle carrier is attached to attract magnetic particles to the particle carrier.

In another embodiment, an apparatus can include a particle carrier attached over the hollow tubular body of a device carrier, where the particle carrier is in the form of a stent that is crimped over the hollow tubular body.

In another embodiment, an apparatus can include a device carrier in the form of a catheter.

In another embodiment, an apparatus can include a pair of rod shaped magnets.

In another embodiment, an apparatus can include at least one magnet made of a rare earth magnetic material.

In another embodiment, an apparatus can include at least one magnetizable guidewire.

In another embodiment, a method is used to preload magnetic particles containing a therapeutic agent onto a medical device for delivering the magnetic particles to a target site in an animal or human subject. The method can include one or more of the following steps:

attaching one or more magnets to a device carrier;

attaching a particle carrier to the device carrier in proximity to the one or more magnets;

inserting the device carrier with the attached particle carrier and the one or more magnets into a suspension containing magnetic particles to magnetically attract a quantity of said magnetic particles to the particle carrier; and

maintaining the device carrier with the attached particle carrier and the one or more magnets in the suspension containing magnetic particles until said quantity of said magnetic particles are preloaded onto the particle carrier.

In another embodiment, a method for preloading magnetic particles containing a therapeutic agent onto a medical device for delivering the magnetic particles to a target site can include the steps of removing the device carrier and attached particle carrier preloaded with said quantity of said magnetic particles from the suspension, and transferring the particle carrier preloaded with said quantity of said magnetic particles from the device carrier to an end of a medical device to be inserted into the animal or human subject, to deliver said quantity of said magnetic particles to the target site.

In another embodiment, a device can include an introducer with a sheath for insertion into the blood vessel. An outer shaft can extend inside the sheath of the introducer, the outer shaft having a distal end. An inner shaft can extend inside the outer shaft, the inner shaft also having a distal end.

The devices can feature a reversibly magnetizable mesh. In one such device, the mesh can include a first end attached to the distal end of the outer shaft and a second end attached to the distal end of the inner shaft, with the mesh surrounding a portion of the inner shaft. The inner shaft can be axially displaceable relative to the outer shaft between a first relative position, in which the magnetizable mesh is radially collapsed, and a second relative position, in which the magnetizable mesh is radially expanded.

In the aforementioned devices, the sheath of the introducer can be a tear-away sheath.

In the aforementioned devices, the devices can include a first lumen for receiving a guidewire.

In the aforementioned devices, the devices can also include a second lumen which is an annular lumen surrounding the first lumen.

In the aforementioned devices, the first lumen can be connected to a first port and the second lumen can be connected to a second port.

Systems are also provided for delivering magnetic particles containing a therapeutic agent to a diseased wall in a blood vessel. In one such system, the system can include a suspension of magnetic particles containing a therapeutic agent. The system can also include a device for delivering the suspension of magnetic particles to the diseased wall in the blood vessel. The device can include an introducer with a sheath for insertion into the blood vessel. An outer shaft can extend inside the sheath of the introducer, the outer shaft having a distal end. An inner shaft can extend inside the outer shaft, the inner shaft also having a distal end.

In the aforementioned systems, one such system can include a device with a magnetizable mesh. The mesh can include a first end attached to the distal end of the outer shaft and a second end attached to the distal end of the inner shaft, the mesh surrounding a portion of the inner shaft. The inner shaft can be axially displaceable relative to the outer shaft between a first relative position, in which the magnetizable mesh is radially collapsed, and a second relative position, in which the magnetizable mesh is radially expanded.

In the aforementioned systems, one such system can include a source for creating a uniform magnetic field.

In the aforementioned systems, one such system can include a source for creating a uniform magnetic field with a permanent dipole magnet or an electromagnetic dipole magnet.

Methods are also provided for delivering magnetic particles to a target site. In one such method, the method includes the steps of generating a uniform magnetic field, and positioning an introducer inside the magnetic field.

In the aforementioned methods, the introducer can define a sheath that contains a magnetizable mesh in a retracted state inside the sheath. The introducer can also be positioned with the mesh located inside the uniform magnetic field to magnetize the mesh.

In the aforementioned methods, the method can include the step of injecting a suspension of magnetic particles into the introducer while the mesh is magnetized in the uniform magnetic field to preload the mesh with the magnetic particles.

In the aforementioned methods, the method can include the step of positioning a distal end of the introducer near the target site.

In the aforementioned methods, the method can include the step of advancing the mesh out of the distal end of the introducer and into proximity of the target site.

In the aforementioned methods, the method can include the step of expanding the mesh in a radially outward direction relative to the introducer to position the mesh adjacent to a structure at the target site so that the magnetic particles contact the structure.

In the aforementioned methods, the method can include the step of maintaining the mesh against the structure to establish adherence of the magnetic particles to the structure.

In the aforementioned methods, the method can include the step of removing the uniform magnetic field.

In the aforementioned methods, the method can include the step of retracting the mesh into the introducer.

In the aforementioned methods, the method can include the step of removing the introducer from the target site.

In the aforementioned methods, the method can include the step of displacing the mesh relative to the introducer to advance the mesh out of a distal end of the sheath.

In the aforementioned methods, the method can include inserting the mesh into a suspension of magnetic particles, with the suspension also being in the magnetic field.

In the aforementioned methods, the method can include the step of targeting the magnetic particles to the mesh and preloading the mesh with the magnetic particles.

In the aforementioned methods, the method can include the step of retracting the mesh back inside the sheath and into the retracted state.

In the aforementioned methods, the method can include positioning the distal end of the sheath near the target site.

In the aforementioned methods, the method can include advancing the mesh out of the distal end of the sheath and into proximity of the target site.

In the aforementioned methods, the method can include expanding the mesh in a radially outward direction relative to the introducer to position the mesh adjacent a structure at a target site so that magnetic particles contact the structure.

### BRIEF DESCRIPTION OF THE DRAWING FIGURES

The following detailed description will be better understood in conjunction with the non-limiting examples and illustrations provided in the following drawing figures, of which:

FIG. 1 is a side view of magnetic targeting device in accordance with one embodiment of the invention;

FIG. 2 is a partially exploded side view of the magnetic targeting device in FIG. 1;

FIG. 3 is a side view of components of the magnetic targeting device of FIG. 1, showing the components in a first operable state;

FIG. 4 is a side view of components of the magnetic targeting device of FIG. 1, showing the components in a second operable state;

FIG. 5 is a side view of the device of FIG. 1 as it could be used in one step of a treatment procedure in accordance with the invention;

FIG. 6 is a side view of the device of FIG. 1 as it could be used in another step of a treatment procedure in accordance with the invention;

FIG. 7 is a side view of the device of FIG. 1 as it could be used in another step of a treatment procedure in accordance with the invention;

FIG. 8 is a side view of the device of FIG. 1 as it could be used in another step of a treatment procedure in accordance with the invention;

FIG. 9 is a side view of the device of FIG. 1 as it could be used in another step of a treatment procedure in accordance with the invention;

FIG. 10 is a side view of magnetic targeting device in accordance with another embodiment of the invention;

FIG. 11 is a truncated perspective view of a device configuration for preloading a medical device with MNP in accordance with the invention;

FIG. 12 is a truncated perspective view of another device configuration for preloading a medical device with MNP in accordance with the invention;

FIG. 13 is a truncated perspective view of another device configuration for preloading a medical device with MNP in accordance with the invention;

FIG. 14 is a truncated perspective view of another device configuration for preloading a medical device with MNP in accordance with the invention;

FIG. 15A is a truncated perspective view of another device configuration for preloading a medical device with MNP in accordance with the invention, with a component of the device shown in a first position prior to transferring the component to a medical device;

FIG. 15B is a truncated perspective view of the device configuration in FIG. 15A, with the component shown in a second position during transfer of the component to a medical device; and

FIG. 15C is a truncated perspective view of the device configuration in FIG. 15A, with the component shown in a third position after the component has been transferred to a medical device.

### DETAILED DESCRIPTION

The challenges experienced with prior magnetic targeting devices and methods are resolved in many respects by an improved device and method for delivering MNP to a treatment site. The improved device and method feature a MTC with a mesh (or other magnetizable component) at the tip of the MTC that is preloaded with MNP prior to being deployed in the artery. To preload the mesh, the MNP are targeted to the mesh in a uniform magnetic field while the mesh is either shielded inside a sheath or deployed in a limited volume of suspended MNP outside of a living organism, human or animal. This preloading step can be done in vivo, if the mesh is shielded inside the sheath, or ex vivo. Once the mesh is preloaded with MNP, the mesh is advanced into the bloodstream and expanded to contact the arterial wall and locally deliver the MNP to the treatment site.

In one embodiment, a device is provided for delivering magnetic particles containing a therapeutic agent to a diseased wall in a blood vessel. The device includes an introducer having a sheath for insertion into the blood vessel. The device also includes a hollow outer shaft extending inside the sheath of the introducer, the hollow outer shaft having a distal end. The device further includes a hollow inner shaft extending inside the outer shaft, the inner shaft having a distal end. Furthermore, the device includes a reversibly magnetizable mesh that includes a first end attached to the distal end of the outer shaft and a second end attached to the distal end of the inner shaft. The mesh surrounds a portion of the inner shaft. The inner shaft is axially displaceable relative to the outer shaft between a first relative position, in which the magnetizable mesh is radially collapsed, and a second relative position, in which the magnetizable mesh is radially expanded.

In another embodiment, a system for delivering magnetic particles containing a therapeutic agent to a diseased wall in a blood vessel includes a suspension of magnetic particles containing a therapeutic agent and a device for delivering the suspension of magnetic particles to the diseased wall. The device includes an introducer, a hollow outer shaft, and a hollow inner shaft. The introducer has a sheath for insertion into the blood vessel. The hollow outer shaft extends inside the sheath of the introducer, and the hollow inner shaft extends inside the outer shaft. The device also includes a magnetizable mesh that includes a first end attached to the distal end of the outer shaft and a second end attached to the distal end of the inner shaft. The mesh surrounds a portion of the inner shaft. The inner shaft is axially displaceable relative to the outer shaft between a first relative position, in which the magnetizable mesh is radially collapsed, and a second relative position, in which the magnetizable mesh is radially expanded. The system further includes a source for creating a uniform magnetic field. The source for creating a uniform magnetic field can include a permanent dipole magnet or an electromagnetic dipole magnet.

In another embodiment, a method for delivering magnetic particles containing a therapeutic agent to a diseased wall in a blood vessel includes the step of generating a uniform magnetic field. The method can also include the step of positioning an introducer inside the magnetic field, the introducer defining a sheath that contains a magnetizable mesh in a retracted state inside the sheath, the introducer being positioned with the mesh located inside the uniform magnetic field to magnetize the mesh. The method can also include the step of injecting a suspension of magnetic particles containing a therapeutic agent into the introducer while the mesh is magnetized in the uniform magnetic field to preload the mesh with the magnetic particles.

The method can also include the step of inserting a distal end of the introducer into a blood vessel and advancing the distal end to a treatment site inside the blood vessel. Moreover, the method can include the step of advancing the mesh out of the distal end of the introducer and into proximity of the treatment site. In addition, the method can include the step of expanding the mesh in a radially outward direction relative to the introducer to position the mesh against a blood vessel wall to be treated at the treatment site so that the magnetic particles contact the blood vessel wall.

The method can also include the step of maintaining the mesh against the blood vessel wall to establish adherence of the magnetic particles to the blood vessel wall. In addition, the method can include the step of removing the uniform magnetic field. The method can also include the step of retracting the mesh into the introducer. Furthermore, the method can include the step of removing the introducer from the treatment site.

In another embodiment, a method for delivering magnetic particles containing a therapeutic agent to a diseased wall in a blood vessel can include the step of generating a uniform magnetic field. The method can also include the step of positioning an introducer inside the magnetic field, the introducer defining a sheath that contains a magnetizable mesh in a retracted state inside the sheath, the introducer being positioned with the mesh located inside the uniform magnetic field to magnetize the mesh. In addition, the method can include the step of displacing the mesh relative to the introducer to advance the mesh out of a distal end of the sheath. Moreover, the method can include the step of inserting the mesh into a suspension of magnetic particles containing a therapeutic agent, with the suspension also being in the magnetic field, to target the magnetic particles to the mesh and preload the mesh with the magnetic particles.

The method can also include the step of retracting the mesh back inside the sheath and into the retracted state. In addition, the method can include the step of inserting the introducer into a blood vessel and advancing the distal end of the sheath to a treatment site inside the blood vessel. Moreover, the method can include the step of advancing the mesh out of the distal end of the sheath and into proximity of the treatment site. Furthermore, the method can include the step of expanding the mesh in a radially outward direction relative to the introducer to position the mesh against a blood vessel wall to be treated at the treatment site so that the magnetic particles contact the blood vessel wall.

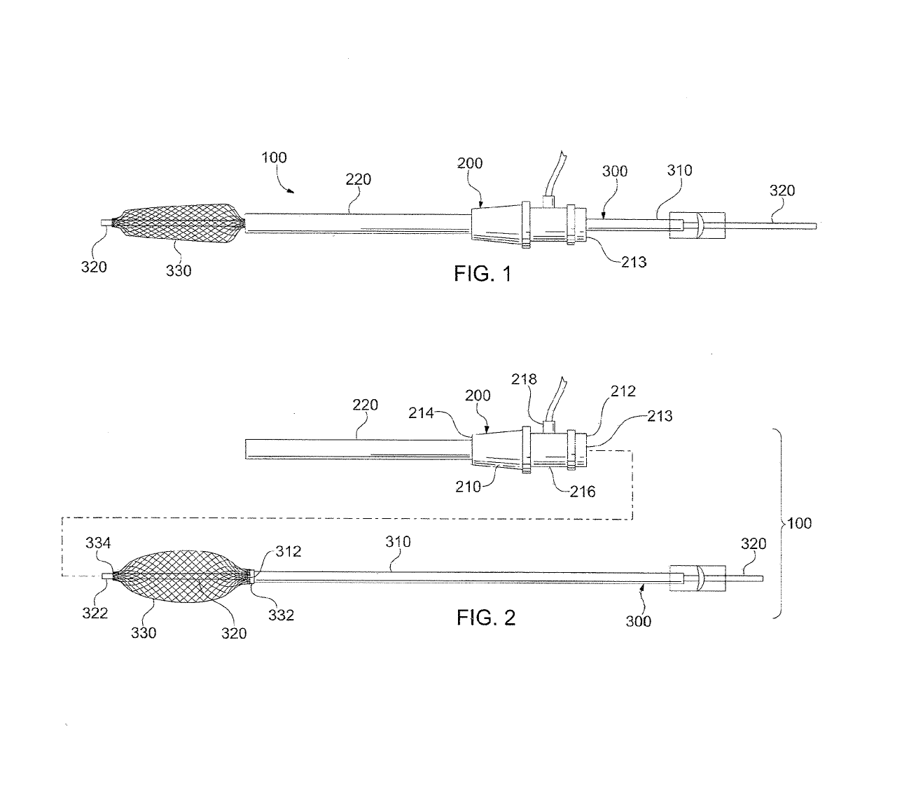

Referring to FIGS. 1 and 2, a device 100 for delivering MNP is shown in accordance with one exemplary embodiment. Device 100 incorporates a MTC that can be inserted into a human or animal subject and navigated to treat one or more areas in the body. In particular, device 100 includes an introducer 200 and a targeting device 300. The targeting device 300 can be pre-loaded with MNP inside introducer 200, and subsequently advanced into a patient to deliver the MNP to a treatment site. For the remainder of this description, examples will assume that the treatment site is an arterial wall. It will be understood, however, that systems, methods and devices in accordance with the invention can be utilized to treat other areas of the body, and are not limited to treating arterial walls.

Introducer 200 includes a body portion 210 having a proximal end 212, a distal end 214 and sidewall 216. Distal end 214 is attached to an elongated sheath 220 that is configured for insertion into an artery. Sidewall 216 includes an injection port 218, which can be a Luer port. Proximal end 212 of body portion 210 defines an opening 213. Opening 213 is adapted to receive targeting device 300, as shown in FIG. 1.

Targeting device 300 includes a hollow outer shaft 310 having a distal end 312 and a hollow inner shaft 320 having a distal end 322. When device 100 is fully assembled, a portion of outer shaft 310 extends inside body portion 210 and sheath 220 of introducer 200. In addition, a portion of inner shaft 320 extends inside outer shaft 310.

Targeting device 300 also includes a magnetizable mesh 330. Mesh 330 has a first end 332 attached to distal end 312 of outer shaft 310. Mesh 330 also has a second end 334 attached to distal end 322 of inner shaft 320. In this arrangement, mesh 330 surrounds a portion of inner shaft 320. Mesh 330 can be made up of strands formed of a reversibly magnetizable material, including but not limited to 430 stainless steel.

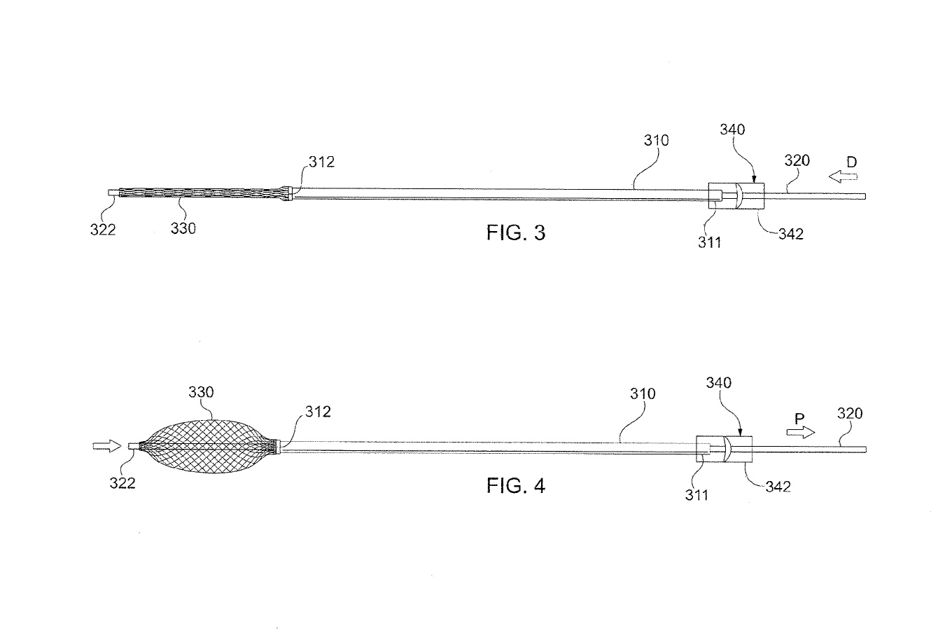

Inner shaft 320 is axially displaceable relative to outer shaft 310 in a telescoping-type arrangement to adjust mesh 330 between a collapsed state and an expanded state. In particular, inner shaft 320 is displaceable relative to outer shaft 310 in a distal direction “D” to a first relative position, shown in FIG. 3. This displacement stretches mesh 330 in a longitudinal direction to a radially collapsed condition as shown. Inner shaft 320 is also displaceable relative to outer shaft 310 in a proximal direction “P” to a second relative position, shown in FIG. 4. This displacement causes mesh 330 to expand radially. Mesh 330 can be collapsed by moving distal end 322 of inner shaft 320 away from distal end 312 of outer shaft, so as to increase the distance between the respective distal ends. Conversely, mesh 330 can be expanded by moving distal end 322 of inner shaft 320 toward distal end 312 of outer shaft, so as to decrease the distance between the respective distal ends.

Targeting device 300 further includes a sleeve 340 attached to a proximal end 311 of outer shaft 310. Inner shaft 320 is axially displaceable through sleeve 340 and into outer shaft 310. Sleeve 340 provides a gripping surface 342 that a user can hold with one hand, while using the other hand to move inner shaft 320 relative to outer shaft 310 to expand or collapse mesh 330.

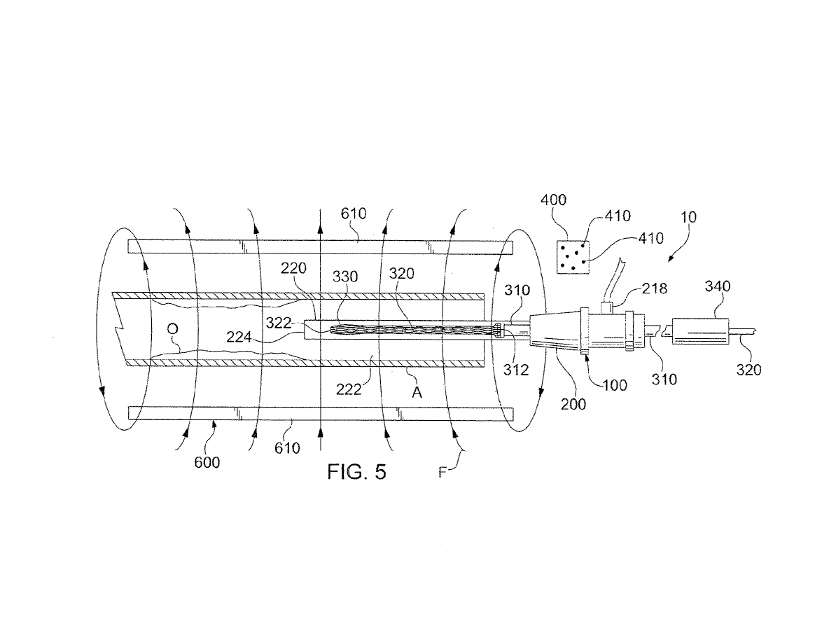

Referring now to FIG. 5, a system 10 for delivering MNP is shown in accordance with the invention. System 10 includes device 100 described previously. In addition, system 10 includes a suspension 400 containing a plurality of MNP 410. Preferably, the volume of suspension 400 is equal to or slightly less than the volume of sheath 220, for reasons that will be explained. System 10 also includes a source for creating a uniform magnetic field 600. Source 600 includes dipole magnets 610 that are configured to generate a 0.1 T uniform magnetic field.

Referring to FIGS. 5-9, a method for delivering MNP containing a therapeutic agent to a treatment site will be described in accordance with one illustrative example. In this example, system 10 is used to deliver MNP to a diseased arterial wall A having a partial obstruction O. Device 100 is preloaded with MNP in vivo in this example. The method of this example can be used as a stand-alone procedure, or as a supplemental procedure that is carried out after other forms of treatment are performed. For example, obstruction O can be treated with a balloon angioplasty or a stent prior to using system 10.

Inner shaft 320 is initially moved to the first relative position, with respect to outer shaft 310, so that mesh 330 is radially collapsed inside sheath 220. To collapse mesh 330, the user can grip sleeve 340 with one hand, grip inner shaft 320 with the other hand, and advance the inner shaft in a pushing motion through outer shaft 310, to move distal end 322 of the inner shaft away from distal end 312 of the outer shaft. Device 100 is then inserted into artery A until sheath 220 is positioned in proximity to obstruction O (FIG. 5). In most cases, this position will be upstream or above the treatment site, with respect to the direction of blood flow. Once sheath 220 is positioned in proximity to obstruction O, a pair of dipole magnets 610 is positioned across the area of artery A containing obstruction O, and across sheath 220, as shown. A uniform magnetic field F of 0.1 T is then established across obstruction O and sheath 220.

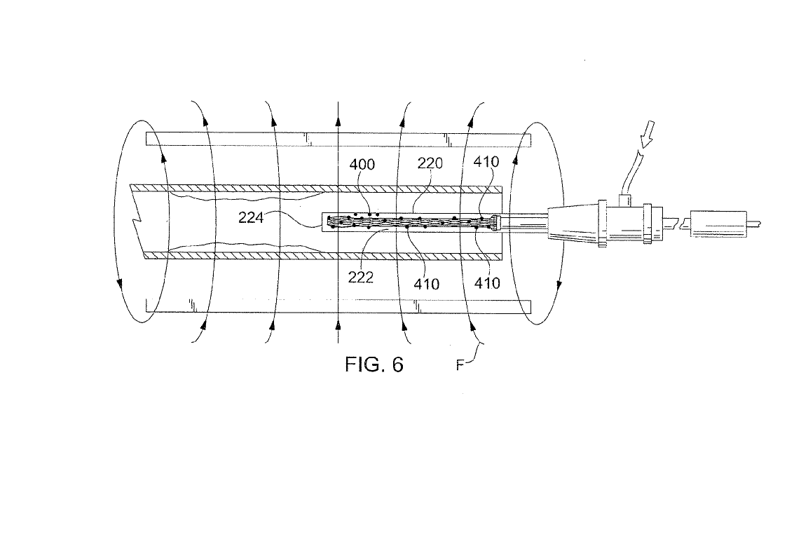

Sheath 220 forms a chamber 222 around mesh 330 to shield the mesh from blood flow in the artery when the mesh is retracted inside the sheath. The space inside sheath 220 that surrounds inner shaft 320 and mesh 330 represents a “free volume”. Suspension 400 containing the plurality of MNP is injected into chamber 222 through injection port 218. Preferably, the user injects a volume of the suspension 400 into sheath 220 that is equal to the free volume in the sheath. This volume is sufficient to surround mesh 330 inside sheath 220 without introducing an excess amount of suspension that can escape out of distal end 224. The injected MNP are immediately attracted and bound to magnetized strands of mesh 330 (FIG. 6). Suspension 400 is left in chamber 222 for a dwell time, for example between about 1 minute and 5 minutes, with magnetic field F remaining in place. Shorter or longer dwell times can also be used.

After the dwell time has expired, suspension 400 is aspirated out of chamber 222 through injection port 218, leaving behind MNP 410 that are bound to mesh 330. Suspension 400 is aspirated out of the chamber 222 while magnetic field F is still in place. Saline can be flushed into chamber 222 as necessary to remove any MNP that are not bound to mesh 330.

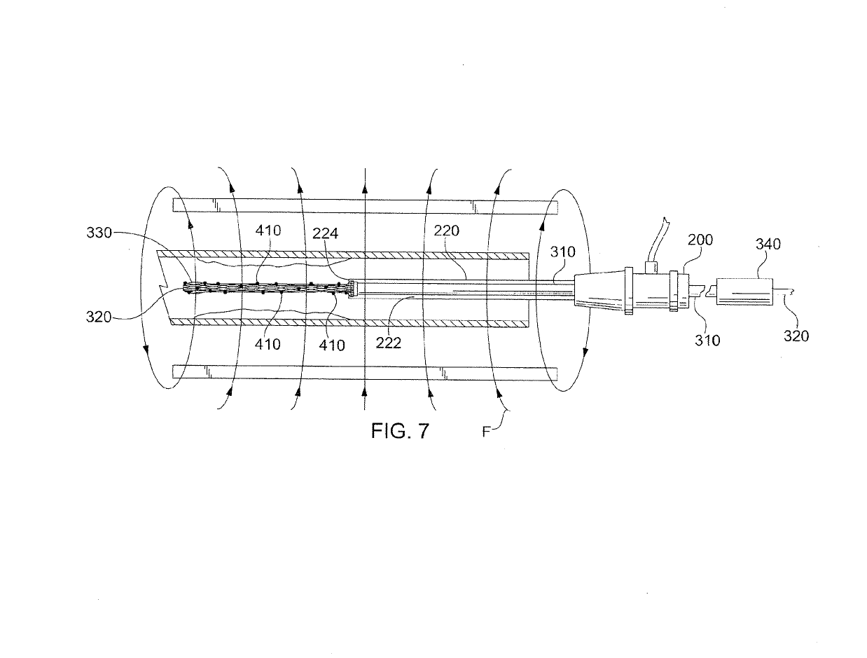

At this stage, mesh 330 is preloaded with MNP and ready to be advanced into the treatment area. To advance mesh 330, the user can grip outer shaft 310 and inner shaft 320 together, and push both through introducer 200 until the mesh emerges out of distal end 224 of sheath 220 (FIG. 7). Mesh 330 is advanced out of sheath 220 with magnetic field F still in place. Depending on the size of the area to be treated, the entire mesh 330 may or may not need to be advanced out of sheath 220.

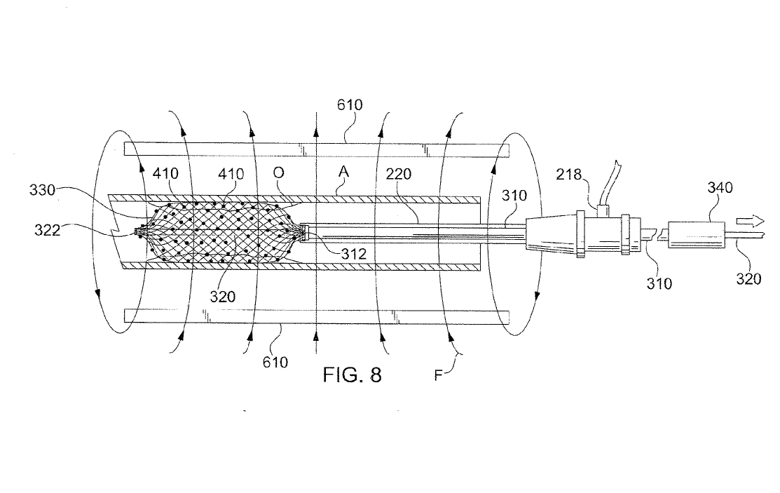

Once the appropriate length of mesh 330 is advanced out of sheath 220, inner shaft 320 is moved to the second relative position, with respect to outer shaft 310, to expand the mesh into contact with obstruction O (FIG. 8). To expand mesh 330, the user can grip sleeve 340 with one hand, grip inner shaft 320 with the other hand, and retract the inner shaft in a pulling motion back into outer shaft 310. This displacement moves distal end 322 of inner shaft 320 toward distal end 312 of outer shaft 310, shortening the length of the mesh so that the mesh radially expands. Once mesh 330 is expanded, the strands of mesh preloaded with MNP 410 contact obstruction O and locally deliver the MNP to arterial wall A.

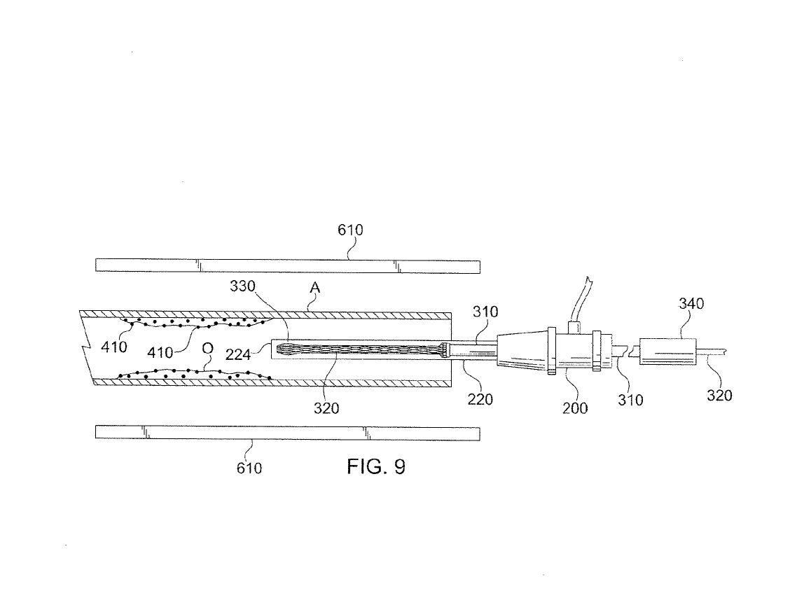

After an appropriate dwell time T, magnetic field F is removed. Dwell time T can be between about 1 minute and 5 minutes. Shorter or longer dwell times can also be used. Upon removal of the magnetic field, MNP 410 that are attracted to the mesh adhere to arterial wall A where the MNP continue to treat the arterial wall. Mesh 330 is then collapsed using the steps described previously. Once mesh 330 is collapsed, inner shaft 320 and outer shaft 310 are retracted back into introducer sheath 220 (FIG. 9). Device 100 can then be moved to another treatment area in the human or animal subject, or withdrawn from the human or animal subject.

Devices similar to device 100 have a number of major advantages. By using a standard introducer design as a sheath for the unexpanded mesh, positioning of the MTC at the site of treatment is simplified. The key guidance event is positioning the distal end of the sheath in proximity to the treatment site. As noted above, it is desirable in most cases to position the distal end of the sheath upstream or above the treatment site.

Devices using an introducer with Luer injection port in accordance with the invention can be customized to optimally accommodate the unexpanded mesh. For example, the introducer sheath can be made in various lengths corresponding to a desired mesh length. The introducer sheath can also incorporate markers so that the sheath's position can be monitored through imaging. For example, the sheath can incorporate x-ray marker bands for localization.

Preloading the mesh with MNP inside an introducer provides the advantage of targeting MNP to the device without “overloading” the device with MNP. As noted earlier, the fluid volume that surrounds the mesh inside the introducer sheath is a fixed volume. A corresponding volume of MNP suspension can be calculated and injected into the sheath, without injecting too much suspension into the sheath. Where the device is being preloaded in vivo, an MNP suspension can be injected into the introducer sheath in the exact volume needed to reach the distal end of the sheath, but no more than this amount, so as to prevent excess suspension from exiting the sheath and releasing excess MNP into the bloodstream before the mesh is deployed. This controlled preloading process prevents MNP from being swept away from the mesh, thereby minimizing biodistribution of MNP that do not adhere to the mesh into the bloodstream.

The MNP already adhere to the mesh before the mesh is exposed to the blood stream. This is particularly beneficial in arteries that have side branches near the treatment area. In prior methods, MNP that were injected into the bloodstream near the mesh could be pulled into the side branches before reaching the mesh. Preloading the mesh with MNP before deploying the mesh in the artery can prevent this from happening. Accordingly, there is no need for mechanisms, such as occlusion balloons, to temporarily halt blood flow in order to prevent loss of MNP.

By preloading MNP inside an introducer sheath in the presence of a magnetic field, it is possible to withdraw the suspension from the sheath after an optimal short period (for example, 1-5 minutes), during which MNP adhesion to the mesh has taken place. It is also possible to flush the introducer with the magnetic field still in place, without displacing mesh adherent particles, but removing non-attached particles from the intra-introducer MNP-suspension. The magnetic field/gradient forces can retain MNP in the mesh. The mesh can subsequently be advanced into the treatment site and mechanically expanded, as previously described.

Once the mesh has contacted the arterial wall in the presence of a magnetic field and is left in place for an optimal time, the magnetic field is then removed. The mesh can then be retracted from the treatment site. At this stage, there will be retention of MNP in the arterial wall. A relatively small number of MNP can escape from the arterial wall or the mesh, prior to retracting the mesh into the introducer. Nevertheless, the number of fugitive MNP that escape from the treatment site will be lower than the number of fugitive MNP that escape when MNP are injected into the bloodstream and magnetically targeted to the mesh. In the improved preloading procedures, most non-adherent MNP are removed from the suspension before the mesh is advanced into the bloodstream. Non-adherent MNP can be removed by intra-introducer flushing inside the introducer sheath, prior to exposing the mesh to the bloodstream.

By minimizing the loss of MNP during delivery, improved devices in accordance with the invention do not need to incorporate occlusion balloons and other mechanisms for controlling the loss of MNP. Therefore, a larger multi-channel catheter device is not necessary to house the occlusion balloons and their control mechanisms, allowing a much smaller MTC to be used. Smaller MTCs can accommodate a wider range of arterial sizes, both large and small, as compared to larger MTCs. In some applications, a guide-wire lumen may not be necessary in the MTC because the MTC can be navigated to the proper location after the introducer sheath is positioned at the treatment site.

Targeting devices like device 300 can be preloaded with MNP in vivo, as described previously. In preferred methods of the invention, the targeting device is preloaded with MNP ex vivo, prior to insertion of the device into the animal or human subject. Ex vivo preloading of MNP has the advantage of allowing optimal time and fluid exposure conditions to permit maximal, controllable uptake of MNP by the magnetizable mesh.

In one ex vivo preloading procedure, introducer 200 and targeting device 300 are inserted into a suspension of MNP that is exposed to a 0.1 T magnetic field. Mesh 330, which is magnetized, remains inside sheath 220 while the sheath is inserted in the suspension. The suspension is then withdrawn into device 100 so that the suspension flows over mesh 330. MNP in the suspension are attracted to mesh 330 and retained by the mesh. The MNP suspension can either be flushed forward or rinsed with water or saline washes, with the 0.1 T field still in place, or rinsed with water or saline washes, to remove MNP that do not adhere to the mesh 330. The introducer device 100 is now preloaded with MNP, and can be used for local delivery of MNP to a treatment site as previously described.

In another ex vivo preloading procedure, inner shaft 310 and mesh 330 are advanced out of distal end 224 of sheath 220 and into a container containing a suspension of MNP. The container is then placed in a 0.1 T magnetic field. Upon insertion of the mesh 330 into the suspension, the MNP are attracted to the magnetized mesh. Mesh 330 remains in the suspension until the mesh is sufficiently preloaded with MNP. Once mesh 330 is preloaded with MNP, the mesh and inner shaft 310 are retracted back into sheath 220, where the MNP remain loaded. The device 100 can then be inserted into a human or animal subject. The treatment site is placed in a 0.1 T magnetic field, and sheath 220 is navigated to a position in proximity to the treatment site. Mesh 330 is advanced out of sheath 220 to the treatment site, and expanded into contact with the arterial wall to locally deliver the MNP to the area in need of treatment.

Two methods for ex vivo preloading of MNP were tested and compared against one another. In a first procedure, “Procedure A”, a MTC was inserted into a suspension of MNP, with a collapsed steel mesh retained inside the distal end of the catheter tip. The test used a 1/10 dilution of a 25 mg/ml suspension of BODIPY-labeled MNP. The suspension of MNP was exposed to a 0.1 T magnetic field for 5 minutes. The suspension was then withdrawn into the catheter shaft over the magnetized steel mesh, where MNP were retained. The catheter was then flushed or rinsed, with the magnetic field still in place, to remove any MNP that did not adhere to the mesh.

In a second procedure, “Procedure B”, a magnetizable mesh was advanced out of the distal end of a MTC tip and submerged in a 1/10 dilution of a 25 mg/ml suspension of BODIPY-labeled MNP. This suspension of MNP was also exposed to a 0.1 T magnetic field for 5 minutes.

The percent mesh uptake results, shown in Table 1 below, were obtained from triplicate measurements (i.e. 3 measurements from the same sample) using fluorometry readings of 96 well plates, 540/570 nm wave length conditions, excitation/emission.

TABLE 1
MTC Uptake by ex vivo Loading - Procedure A (MNP capture in 0.1T
within the introducer volume) vs. Procedure B (MNP capture in 0.1T with
MTC tip extruded past the tip of the introducer into a small concentrated
volume of MNP) Endpoint % fluorescent MNP depleted

| | Capture efficiency-% | | |
|---|---|---|---|
| Procedure A | 33.9 | 36.1 | 29.9 |
| Procedure B | 83.2 | 79.7 | 84.1 |

As shown in Table 1, the average percent uptake of MNP using Procedure A was 33.3%, while the average percent uptake of MNP using Procedure B was 82.3%. These results demonstrate that, while both procedures are effective in preloading MNP, Procedure B results in a much more efficient MNP uptake than Procedure A.

The kinetics of Procedure B were studied using a 1/10 dilution of a 25 mg/ml suspension of BODIPY-labeled MNP, and a 0.1 T magnetic field. Samples were taken at different time intervals and assayed for MNP fluorescence. Table 2 summarizes the capture efficiency that was measured in triplicate after magnetic field exposure times of 2 minutes, 5 minutes, 10 minutes and 15 minutes.

TABLE 2
Studies of 2.5 mg MNP dispersed in 4 ml Uptake (%) per depletion assay

| Timepoint | Capture efficiency-% | | |
|---|---|---|---|
| 2 min | 78.7 | 83.0 | 79.3 |
| 5 min | 86.0 | 86.6 | 84.3 |
| 10 min | 90.7 | 90.2 | 91.3 |
| 15 min | 93.1 | 93.2 | 92.5 |
| Recovered from mesh (% of original) | 86.8 | 80.2 | 90.9 |

As can be seen, the average MTC uptake was approximately 80% after 2 minutes, approximately 91% after 10 minutes, and approximately 93% after 15 minutes. After the magnetic field was discontinued, almost all of the MNP were recovered from the MTC. These results further confirm the efficiency of Procedure B and suggest that a sufficient uptake of MNP can be achieved with a dwell time as little as 5 minutes or less.

In a separate experiment, uptake of MNP was studied using Procedure B, with a maximal loading of MNP (25 mg) dispersed for magnetic uptake in 4 ml of water, sufficient to cover a steel mesh. A steel mesh made of 430 stainless steel was attached at one end to the distal end of an outer shaft, similar to mesh 330 and outer shaft 320. The other end of the mesh was attached to the distal end of an inner shaft slidably received in the outer shaft, similar to mesh 330 and inner shaft 320. The inner shaft, outer shaft and mesh, hereinafter referred to as an “expansion stick”, was inserted into a catheter introducer. The mesh was extruded from the end of the introducer and submerged in the MNP suspension, which was observed to have an opacity. The suspension was placed in a 0.1 T magnetic field using a dipole magnet or C-core dipole configuration. As the MNP were attracted to the mesh, the opacity diminished and the suspension began to visibly clarify after 30 seconds of exposure time. After two minutes of exposure time, the MNP suspension was almost clear. 100 ul samples were taken and appropriately diluted to quantitate the depletion of the MNP suspension by attraction to the magnetized mesh. Capture efficiency data resulting from depletion of MNP from suspension are shown below in Table 3.

TABLE 3
25 mg MNP in 4 ml--% depleted

| Timepoint | Capture efficiency-% | | |
|---|---|---|---|
| 2 min | 88.5 | 89.8 | 91.1 |
| 5 min | 92.0 | 93.1 | 92.0 |
| 10 min | 96.2 | 93.3 | 90.6 |
| 15 min | 94.8 | 96.4 | 96.6 |

Table 3 shows that between 88% and 91% of particles were taken up by two minutes, with increased uptake at later time points, thus establishing the efficiency of an optimal preloading technique in which the mesh is deployed in a suspension, followed by 0.1 T magnetic field exposure. After magnetic field exposure, the mesh was deployed in water and the percent recovery of MNP from the original 25 mg in 4 ml was measured. In triplicate assays after the magnetic field was discontinued, the recovery of MNP ranged from 79% to 86%.

In another experiment, Procedure B was tested to quantitate the uptake of MNP and observe the ability of the mesh to retain preloaded MNP after the mesh is removed from the magnetic field. As in the previous experiment, this experiment used Procedure B with the maximal loading of MNP (25 mg) dispersed for magnetic uptake in 4 ml of water, sufficient to cover the steel mesh. The 430 steel mesh on the end of an expansion stick was extruded from the end of the introducer and submerged into a test tube containing the MNP suspension. The suspension was then placed in the 0.1 T field. The MNP suspension began to visibly clarify after 30 seconds. After two minutes, the suspension was greatly reduced in opacity—almost clear. 100 ul samples of the suspension were taken and appropriately diluted to quantitate the depletion of the MNP suspension by attraction to the magnetized mesh.

After two minutes, the mesh was collapsed by forward movement of the expansion stick shaft. The mesh was then withdrawn into the sheath of the introducer while remaining in the presence of a 0.1 T magnetic field. Following this, the introducer, with the mesh contained in the sheath under hydraulic seal, was taken out of the test tube and transferred into 4 ml of distilled water, which was not in the presence of a magnetic field. The introducer was observed for one minute. No leakage was observed during the observation period, and no fluorescence could be detected. The introducer tip was then placed back into the test tube exposed to the 0.1 T magnetic field. The expansion stick was advanced out of the introducer shaft until the mesh protruded from the end of the introducer. The mesh was then expanded in the 4 ml volume in the glass test tube with the 0.1 T magnetic field still present. The 4 ml volume appeared clear, and no particle release was observed or quantitated with fluorescence. The test tube was then removed from the 0.1 T magnetic field. Within 10 seconds, the clear water in the test tube became densely brown in color, providing visual indication of a significant release of MNP from the mesh. The uptake, release and recovery data are shown below in Table 4.

TABLE 4
25 mg MNP in 4 ml-Data in percentages per fluorescent assays, in
triplicate (540/570 nm)

| | | | |
|---|---|---|---|
| 2 min-uptake by mesh | 88.5% | 89.8% | 91.1% |
| Release and recovery | 75.1% | 73.7% | 67.1% |
| of captured MNP with | | | |
| deployment and | | | |
| discontinuation of | | | |
| magnetic field. | | | |

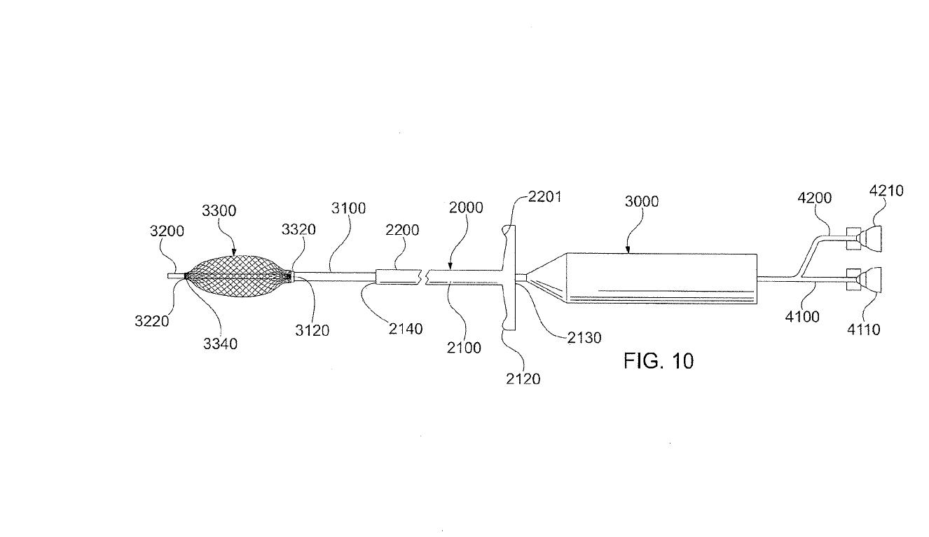

Referring now to FIG. 10, an alternative device 1000 for delivering MNP is shown in accordance with another exemplary embodiment. Device 1000 can include any or all of the same components included in device 100. Some of the same features present in device 100 and device 1000 will be omitted in this section for brevity.

Like device 100, device 1000 incorporates a MTC that can be inserted into a human or animal subject and navigated to treat one or more areas in the body. In particular, device 1000 includes an introducer 2000 and a targeting device 3000. The targeting device 3000 can be pre-loaded with MNP inside introducer 2000, and subsequently advanced into a patient to deliver the MNP to a treatment site.

Introducer 2000 includes a body portion 2100 having a proximal end 2120, a distal end 2140, and an elongated sheath 2200 extending between the proximal and distal ends that is configured for insertion into an artery. Introducer 2000 includes an injection port, which can be a Luer port. Proximal end 2120 of body portion 2100 defines an opening 2130. Opening 2130 is adapted to receive targeting device 3000.

A pair of wings or pull-tabs 2201 extend radially outwardly from sheath 2200. Sheath 2200 is formed of a material that can tear longitudinally along the length of the sheath in response to outward force applied to each of the pull tabs 2201. Once introducer 2000 and targeting device 3000 are inserted in a subject, outward force can be applied to pull-tabs 2201 to tear or split sheath 2200 into two halves that can be removed from around the targeting device while keeping the targeting device inside the subject.

Targeting device 3000 includes a hollow outer shaft 3100 having a distal end 3120 and an inner shaft 3200 having a distal end 3220. When device 1000 is fully assembled, a portion of outer shaft 3100 extends inside sheath 2200 of introducer 2000. In addition, a portion of inner shaft 3200 extends inside outer shaft 3100.

Targeting device 3000 also includes a magnetizable mesh 3300. Mesh 3300 has a first end 3320 attached to distal end 3120 of outer shaft 3100. Mesh 3300 also has a second end 3340 attached to distal end 3220 of inner shaft 3200. In this arrangement, mesh 3300 surrounds a portion of inner shaft 3200. Mesh 3300 can be made up of strands formed of a reversibly magnetizable material, including but not limited to 430 stainless steel.

Inner shaft 3200 is axially displaceable relative to outer shaft 3100 in a telescoping-type arrangement to adjust mesh 3300 between a collapsed state and an expanded state in the same manner described above with respect to mesh 330.

Devices and systems in accordance with the invention can include one or more ports and lumen, each dedicated to a specific function. In device 1000, for example, the device includes a first lumen 4100 and a second lumen 4200. First lumen 4100 connects with the interior of introducer and is configured to allow passage of a guidewire through the introducer. Second lumen 4200 is an annular lumen that surrounds first lumen 4100 and can be used to flush or rinse out device 1000. First lumen 4100 is connected to a first port 4110 and second lumen 4200 is connected to a second port 4210.

Devices and methods in accordance with the invention can include various kinds of multicomponent magnetic devices and methods for preloading implants with magnetizable materials. Preloading implantable devices that are either used surgically or as part of an intervention, is optimally carried out prior to deployment or implantation. This preloading can be utilized to preload virtually any type of stent or interventional device with cells containing magnetic particles (thereby rendering the cells magnetically responsive) or magnetic particles containing therapeutic agents or imaging compounds. Devices and methods in accordance with the invention can include the use of any of the following: 1) iron oxide containing particles, made from either biodegradable or nondegradable materials, that are prepared, of any dimensions needed for in vivo use, as either nanoparticles or microparticles; 2) nanoparticles or microparticles containing either imaging compounds and/or therapeutic agents (that could be pharmaceuticals, viral vectors, peptides or proteins); 3) any implantable device, such as a stent, composed of any material, metallic or polymeric/bioresorbable, crimped onto or otherwise connected to the end of a balloon tip catheter; 4) a magnetizable guidewire (ideally high carbon steel, such as “music wire”) that is inserted into a catheter tip lumen, occupying the distal part of the shaft, to enhance the magnetic attraction to both particles and the magnetic source; 5) rod shaped diametrically magnetized magnets; 6) a magnetic source, ideally a neodymium rare earth magnetic or an electromagnetic material, either as a single source or part of a dipole, as an external dipole, or as rods inserted into the lumen of the distal catheter shaft.

Preloading of a device such as a stent can be achieved with the follow steps: 1) Cells are preloaded with magnetic particles at an ideal density, or if particles alone are used, these are formulated in a suspension at an ideal density; 2) A catheter tip with a crimped stent is inserted into the magnetizable cell or particle suspension in an optimally small volume tube; 3) This tube-stent configuration is then secured against the surface of the magnet, with an optimal magnetic field, for an ideal period (e.g. 5 minutes for cell adhesion to the stent), and a fraction of the cells or particles are attracted to the surface of the stent. Ideally this step is repeated with a fresh cell or particle suspension using the opposite magnet in a dipole to optimally coat all surfaces of the stent. The stent-tube configuration is then removed from the magnetic field and is inserted into a protective sheath to enable deployment of the preloaded stent in an artery. 4) Alternatively, rod shaped, diametrically magnetized magnets can be inserted into a catheter shaft lumen (ideally distally where the stent would reside) of an appropriate diameter, and the stent partially crimped externally over the magnetic region; this stent, catheter composite can then be placed in a cell suspension with MNP-preloaded cells, that are magnetically attracted and thus attached to the stent-tube-rod magnet complex. Following cell loading or particle loading, the pre-crimped stent can then be slid from the catheter-rod segment onto a collapsed balloon at the tip of an angioplasty catheter for arterial expansion and permanent deployment.

A series of experiments have been carried out demonstrating the feasibility of preloading implants or implantable devices in accordance with the invention. These experiments are summarized below.

### Experiment 1

This study examined magnetic preloading of iron oxide containing biodegradable magnetic nanoparticles (MNP). A 316L steel stent (nonmagnetic) was crimped onto the end of a catheter that was steel reinforced with magnetically responsive steel, placed into a suspension of MNP, 2.5 mg/ml in a glass test tube, that was then placed in between a dipole, permanent magnet, with a field of 0.1 T. The MNP suspension clarified in 2.5 minutes with dense particle coverage over the region of the stent, due to the underlying steel-magnetic catheter tubing, with a non-stent/steel tube containing control suspension not. When the catheter was removed from the field, the bound particles were released.

### Experiment 2

The next series of experiments confirmed the benefits of inserting rod shaped magnets, rare earth in composition, 1/16″ in diameter & ¼″ in length. Two of these magnets were inserted into the shaft of a cardiac catheter segment and placed in a MNP suspension (2.5 mg/ml as above), without a stent crimped onto the surface. The result was rapid clarification of the MNP suspension, in only one minute, compared to control.

### Experiment 3

Additional studies demonstrated the benefits of using a magnetizable steel guidewire inserted into a catheter or introducer shaft, that would be become magnetized in a uniform field, and thereby attract MNP or MNP-loaded cells to a stent crimped onto the surface of the catheter guidewire composite. In these experiments, a non-magnetic (316 steel stent) was crimped onto the end of an introducer rod, with a steel guidewire (that was magnetizable), and then inserted into a 2.5 mg/ml MNP suspension in water, and placed in the 0.1 T field as above. The solution clarified in 3 minutes, and the removed stent showed obvious MNP deposition its surface, compared to the unimplanted state, confirming preloading of MNP onto the non-magnetic steel stent with this technique.

### Experiment 4

Other studies examined different configurations for preloading stents. In these studies, rod shaped rare earth magnets (diametrically magnetized) were configured in a six magnet array. The array of magnets was designed to be placed around the tip of a balloon angioplasty catheter, after which a stent could be crimped on top of the magnets for localized MNP or cell-MNP loading with a strong field (ca 400 gauss). Following preloading, the stent was designed to be easily removed and transferred to another catheter, or the magnets could be quickly removed through exposure to a steel rod. The preloaded stent could then be inserted into an artery to be treated and deployed at a specific site.

### Experiment 5

A series of experiments were carried out using high carbon steel wire (“Music Wire”) that could become permanently magnetized with exposure to a magnetic field. The wire was then inserted into the lumen of angioplasty catheters and used as a guidewire that could enhance the attraction of MNP or cells loaded with MNPs to a non-magnetic stent crimped into the end of a balloon tip catheter. A successful experiment was completed using this configuration in vitro, exposing the steel wire (premagnetized), catheter-balloon tip, stent (non-magnetic) construct to a single pole of a dipole, with a measure field strength of 4000 Gauss.

### Experiment 6

In this experiment, a stent was connected directly onto rod shaped rare earth magnets for preloading with MNP or MNP-cells, with transfer to a balloon tip catheter. The stent was placed onto 1/16″ rod magnets. The magnets were diametrically magnetized and inserted in the shaft of the stent, which was partially crimped onto the rod shaped magnets. The stent and magnets were capable of being preloaded with MNP ex vivo in a cell culture incubation. The end of one magnet was then attached to the end of a balloon tip catheter, and the stent was partially slid and transferred onto the surface of the balloon of the balloon tip catheter. Once transferred onto the balloon, the partially crimped stent was expandable with the balloon to deploy the stent.

### Experiment 7

In this experiment, rod-shaped rare earth magnets were inserted into polyurethane grade medical tubing that simulated a catheter tip. A stent was crimped over the exterior of the catheter tip, simulating a device for MNP/cell-MNP preloading. The stent was then slide-transferred to a balloon angioplasty catheter tip, where it could be deployed as an interventional device. The composite stent-catheter with rod magnets can be incubated in a suspension of cells preloaded with MNP that would be magnetically attracted to the stent and surface of the rod due to the magnetic forces of the rare earth magnets within the lumen of the catheter tip. The same approach could be used for a suspension of MNP or magnetic microparticles. After an optimal period of time, the stent-catheter-rod magnet composite can be removed from the MNP-cell/or MNP suspension and juxtaposed to the tip of a balloon tip angioplasty catheter, at which time the stent with cells attached can be transferred onto the balloon by sliding the stent onto the balloon.

### Experiment 8

This study examined in vitro magnetic cell preloading with a stent mounted on a catheter with magnetic rods in the catheter lumen. Bovine aortic endothelial cells (BAEC) labeled with calceine—to document viability & enable fluorescent imaging (ca. 100,000 cells per ml) were preloaded with magnetic nanoparticles. The cells were then incubated with the stent-catheter-magnet assembly described in Experiment 7, for a period of 3 hours. Following this, the stent-catheter segments were removed and subjected to fluorescent microscopy using filters to image the green fluorescent calcein-positive cells. Images showed that nonmagnetic exposure of the stent and the catheter (no rod magnets used) resulted in no detected cells, whereas the magnetic assembly (2 diametrically magnetized, 1/16″ rod shaped rare earth magnets) resulted in dense cell coverage of the stent wires and underlying catheter shaft with BAEC.

### Observations from Experiments

The following steps can be used in vivo either in an experimental animal or human subject:
Inserting rod shaped magnets into a catheter tube, and crimping a stent over the exterior of the catheter tube, over the region where the magnets are inserted;
Incubating the composite stent-catheter with rod magnets in a suspension of cells preloaded with MNP;
Transferring the stent with preloaded cells to a balloon tip catheter;
Catheterizing a blood vessel to be treated with the balloon tip catheter; and
Deploying the stent in the blood vessel to deliver the preloaded cells to the blood vessel wall.

The foregoing technique can enable magnetic cell delivery using virtually any type of stent, not only steel stents, but any alloy and non-metallic stents and scaffolds. This approach represents a major advance over previous approaches that require the use of magnetizable steel stents and involved in vivo only stent targeting in magnetic fields generated by dipoles positioned across the subject with a deployed stainless steel stent.

Although some of the experiments described above involved stents with openings between wires, it is contemplated that a so-called “covered stent” could also be used. The covering between the wires on the stent could trap even more cells than those trapped on the framework. Thus, the cells that pass through the stent framework openings could reside on the covering, ready for delivery to the arterial wall when the stent is deployed. The covering could be either non-degradable or bio-resorbable.

FIG. 11 shows another device configuration 700 for preloading a medical device with MNP or cells loaded with MNP in accordance with the invention. Device configuration 700 includes a “device carrier” in the form of a hollow tubular shaft 710. Hollow tubular shaft 710 can take many forms, such as the tip of a catheter. A pair of rod shaped magnets 720 are inserted inside tubular shaft 710. Magnets 720 are composed of a rare earth element and have a diameter of about 1/16 inch. A “particle carrier” in the form of a non-magnetic stent 730 made of 316 steel is crimped over an end portion 712 of tubular shaft 710. Device configuration 700 can be inserted into a suspension of MNP or cells loaded with MNP, such as a 2.5 mg/ml suspension, to attract a quantity of therapeutic MNP or cells to tubular shaft 710 and stent 730.

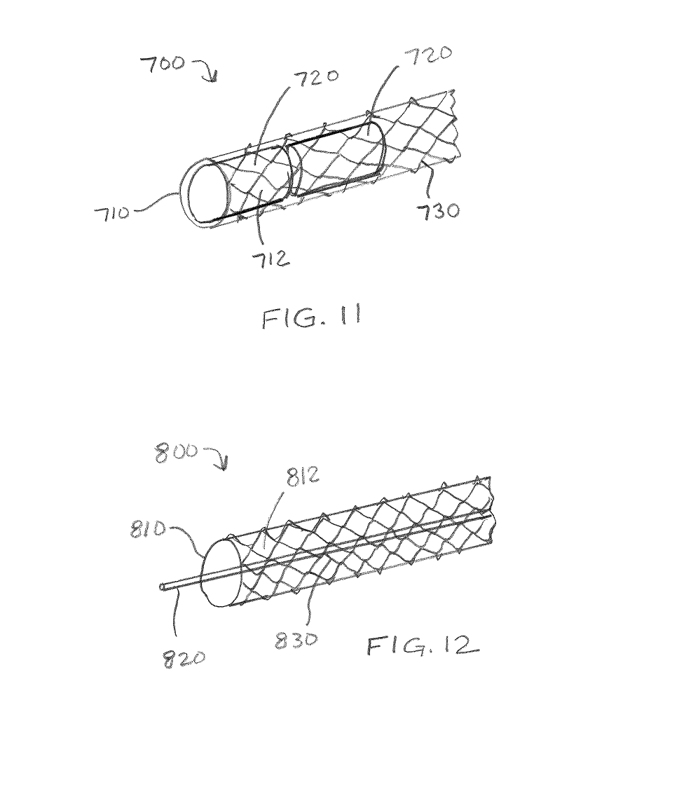

FIG. 12 shows another device configuration 800 for preloading a medical device with MNP or cells loaded with MNP in accordance with the invention. In this example, device configuration 800 includes a device carrier in the form of a hollow tubular shaft 810, which again may the tip of a catheter. Tubular shaft 810 surrounds a magnetizable steel guidewire 820 inserted into the tubular shaft. A particle carrier in the form of a non-magnetic stent 830 made of 316 steel is crimped over an end portion 812 of tubular shaft 810. Tubular shaft 810 with guidewire 820 and stent 830 can be inserted into a suspension of MNP or cells loaded with MNP, such as a 2.5 mg/ml suspension. The suspension of MNP or cells loaded with MNP and device configuration 800 can then be placed in a uniform magnetic field (0.1 T). Once placed in the field, guidewire 820 becomes magnetized and attracts a quantity of MNP or cells loaded with MNP to tubular shaft 810 and stent 830. FIG. 13 shows another device configuration 900 for preloading a medical device with MNP or cells loaded with MNP in accordance with the invention. Device configuration 900 includes a device carrier in the form of a balloon angioplasty catheter 910. An array of six rod shaped magnets 920 (three of which are visible, the other three being obstructed from view, but identical in configuration) are arranged in a surrounding fashion around catheter 910. Magnets 920 are composed of rare earth and have a diameter of about 1/16 inch. A particle carrier in the form of a non-magnetic stent 930 made of 316 steel is crimped over an end portion 912 of catheter 910. In this arrangement, stent 930 is subjected to a strong field (e.g. 400 gauss) which can be used for localized preloading of MNP or cells loaded with MNP onto the stent. Following preloading, the preloaded stent 930 can be removed from catheter 910 and transferred to another catheter. In addition, or in the alternative, magnets 920 can be removed through exposure to a steel rod. The preloaded stent 930 can then be inserted directly into an artery and deployed at a specific site for treatment.

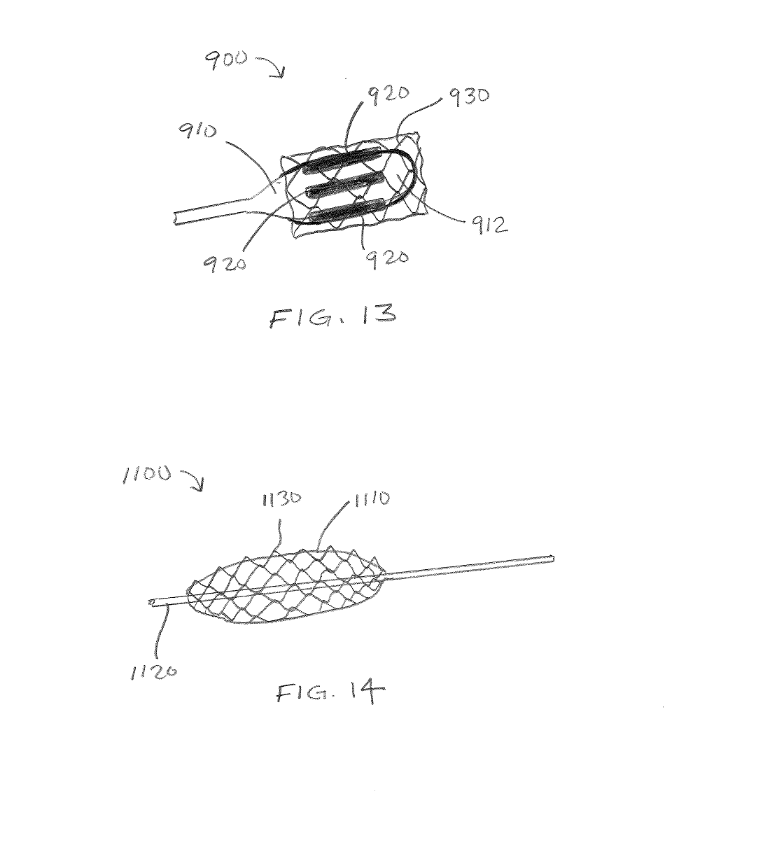

FIG. 14 shows another device configuration 1100 for preloading a medical device with MNP or cells loaded with MNP in accordance with the invention. In this example, device configuration 1100 includes a device carrier in the form of an angioplasty catheter 1110. Catheter 1110 surrounds a high carbon steel wire 1120 (or “music wire”) that can be permanently magnetized with exposure to a magnetic field. Once wire 1120 is magnetized, it is inserted into the lumen of catheter 1110 which is surrounded by a particle carrier in the form of a stent 1130 crimped over the catheter. Catheter 1110 can then be inserted into a suspension of MNP or cells loaded with MNP, such as a 2.5 mg/ml suspension. The catheter-stent-wire construct is exposed to a single pole of a dipole, for example with a field strength of 4000 gauss. The strong attraction between wire 1120 and the magnet holds the catheter tip in a stable manner next to the magnet while a quantity of MNP are drawn to the magnet and stent.

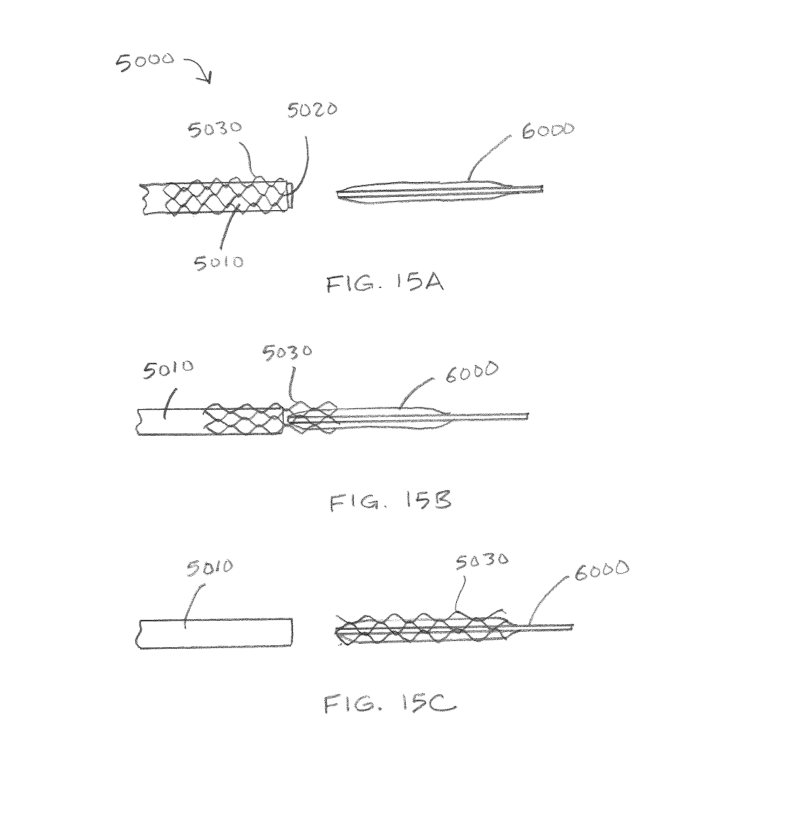

FIGS. 15A-15C show another device configuration 5000 for preloading a medical device with MNP or cells loaded with MNP in accordance with the invention. In this example, device configuration 5000 includes a device carrier in the form of a hollow tubular shaft 5010, which again may the tip of a catheter. A pair of rod shaped magnets 5020 similar or identical to magnets 720 are inserted inside tubular shaft 5010. The magnets 5020 are composed of rare earth and have a diameter of about 1/16 inch.

Referring to FIG. 15A, a particle carrier in the form of a non-magnetic stent 5030 made of 316 steel is crimped over an end portion 5012 of tubular shaft 5010. Tubular shaft 5010 with magnets 5020 and stent 5030 can be inserted into a suspension of MNP or cells loaded with MNP, such as a 2.5 mg/ml suspension, to preload the stent with a quantity of MNP or cells loaded with MNP. After stent 5030 is preloaded with MNP or cells loaded with MNP, the stent can be slid partially off of tubular shaft 5010 as shown in FIG. 15B. Stent 5030 can be slid off of tubular shaft 5010 using conventional instruments and techniques. The portion of stent 5030 that is slid off of tubular shaft 5010 can be aligned with the tip or end of a balloon angioplasty catheter 6000, and subsequently advanced over a collapsed balloon portion of the balloon angioplasty catheter, as shown. Stent 5030 is advanced over balloon angioplasty catheter 6000 until the preloaded stent is completely removed from tubular shaft 5010, as shown in FIG. 15C. At this stage, balloon angioplasty catheter 6000 with preloaded stent 5030 can be inserted into an animal or human subject and deployed at a treatment area to deliver MNP or cells loaded with MNP to the treatment site.

Although the invention is illustrated and described herein with reference to specific embodiments, the invention is not intended to be limited to the details shown. The specific embodiments described herein are provided by way of example only. Numerous variations, changes and substitutions will occur to those skilled in the art without departing from the scope of the invention. For example, the description refers primarily to the use of MNP. Nevertheless, it is envisioned that magnetic particles that are not of nano-particle size could be used in the systems and methods of the invention. Accordingly, it is intended that the appended claims cover all such variations as fall within the scope of the invention.

What is claimed:

1. An apparatus for preloading magnetic particles onto a medical device for delivering the magnetic particles to a target site, the apparatus comprising:
a device carrier comprising a hollow tubular body;
a particle carrier attached over the hollow tubular body of the device carrier; and
at least one magnet inserted into the hollow tubular body of the device carrier, the at least one magnet extending inside a section of the tubular body over which the particle carrier is attached to attract magnetic particles to the particle carrier.

2. The apparatus of claim 1, wherein the particle carrier attached over the hollow tubular body of the device carrier comprises a stent that is crimped over the hollow tubular body.

3. The apparatus of claim 1, wherein the device carrier comprises a catheter.

4. The apparatus of claim 1, wherein the at least one magnet comprises a pair of rod shaped magnets.

5. The apparatus of claim 1, wherein the at least one magnet is made of a rare earth element.

6. The apparatus of claim 1, wherein the at least one magnet comprises a magnetizable guidewire.

7. A method for preloading magnetic particles containing a therapeutic agent onto a medical device for delivering the magnetic particles to a target site in an animal or human subject, the method comprising the steps of:
attaching one or more magnets to a device carrier;
attaching a particle carrier to the device carrier in proximity to the one or more magnets;
inserting the device carrier with the attached particle carrier and the one or more magnets into a suspension containing magnetic particles to magnetically attract a quantity of said magnetic particles to the particle carrier; and
maintaining the device carrier with the attached particle carrier and the one or more magnets in the suspension containing magnetic particles until said quantity of said magnetic particles are preloaded onto the particle carrier.

8. The method of preloading magnetic particles of claim 7, further comprising the steps of:
removing the device carrier and attached particle carrier preloaded with said quantity of said magnetic particles from the suspension; and
transferring the particle carrier preloaded with said quantity of said magnetic particles from the device carrier to an end of a medical device to be inserted into the animal or human subject, to deliver said quantity of said magnetic particles to the target site.

9. A device for delivering magnetic particles containing a therapeutic agent to a diseased wall in a blood vessel, the device comprising:
an introducer comprising a sheath for insertion into the blood vessel;
an outer shaft extending inside the sheath of the introducer, the outer shaft having a distal end;
an inner shaft extending inside the outer shaft, the inner shaft having a distal end; and
a reversibly magnetizable mesh,
the mesh comprising a first end attached to the distal end of the outer shaft and a second end attached to the distal end of the inner shaft, the mesh surrounding a portion of the inner shaft,
the inner shaft being axially displaceable relative to the outer shaft between a first relative position, in which the magnetizable mesh is radially collapsed, and a second relative position, in which the magnetizable mesh is radially expanded.

10. The device of claim 9, wherein the sheath of the introducer is a tear-away sheath.

11. The device of claim 9, further comprising a first lumen for receiving a guidewire.

12. The device of claim 11, further comprising a second lumen which is an annular lumen surrounding the first lumen.

13. The device of claim 12, wherein the first lumen is connected to a first port and the second lumen is connected to a second port.

14. A system for delivering magnetic particles containing a therapeutic agent to a diseased wall in a blood vessel, the system comprising:
A. a suspension of magnetic particles containing a therapeutic agent;
B. a device for delivering the suspension of magnetic particles to the diseased wall in the blood vessel, the device comprising:
i. an introducer comprising a sheath for insertion into the blood vessel;
ii. an outer shaft extending inside the sheath of the introducer, the outer shaft having a distal end;
iii. an inner shaft extending inside the outer shaft, the inner shaft having a distal end; and
iv. a magnetizable mesh, the mesh comprising a first end attached to the distal end of the outer shaft and a second end attached to the distal end of the inner shaft, the mesh surrounding a portion of the inner shaft, the inner shaft being axially displaceable relative to the outer shaft between a first relative position, in which the magnetizable mesh is radially collapsed, and a second relative position, in which the magnetizable mesh is radially expanded; and
C. a source for creating a uniform magnetic field.

15. The system of claim 14, wherein the source for creating a uniform magnetic field comprises a permanent dipole magnet or an electromagnetic dipole magnet.

16. A method for delivering magnetic particles to a target site, the method comprising the steps of:
generating a uniform magnetic field;
positioning an introducer inside the magnetic field, the introducer defining a sheath that contains a magnetizable mesh in a retracted state inside the sheath, the introducer being positioned with the mesh located inside the uniform magnetic field to magnetize the mesh;
injecting a suspension of magnetic particles into the introducer while the mesh is magnetized in the uniform magnetic field to preload the mesh with the magnetic particles;
positioning a distal end of the introducer near the target site;
advancing the mesh out of the distal end of the introducer and into proximity of the target site;
expanding the mesh in a radially outward direction relative to the introducer to position the mesh adjacent to a structure at the target site so that the magnetic particles contact the structure;
maintaining the mesh against the structure to establish adherence of the magnetic particles to the structure;
removing the uniform magnetic field;
retracting the mesh into the introducer; and
removing the introducer from the target site.

17. A method for delivering magnetic particles to a target site, the method comprising the steps of:
generating a uniform magnetic field;
positioning an introducer inside the magnetic field, the introducer defining a sheath that contains a magnetizable mesh in a retracted state inside the sheath, the introducer being positioned with the mesh located inside the uniform magnetic field to magnetize the mesh;
displacing the mesh relative to the introducer to advance the mesh out of a distal end of the sheath;
inserting the mesh into a suspension of magnetic particles, with the suspension also being in the magnetic field, to target the magnetic particles to the mesh and preload the mesh with the magnetic particles;
retracting the mesh back inside the sheath and into the retracted state;
positioning the distal end of the sheath near the target site;
advancing the mesh out of the distal end of the sheath and into proximity of the target site; and
expanding the mesh in a radially outward direction relative to the introducer to position the mesh adjacent a structure at the target site so that the magnetic particles contact the structure.
